(19) United States
(12) Patent Application Publication
(10) Pub. No.: US 2024/0091118 A1
(43) Pub. Date: Mar. 21, 2024

(54) THERMALLY STABLE, MINERAL OIL-FREE W/O EMULSION

(71) Applicant: BEIERSDORF AG, Hamburg (DE)

(72) Inventors: Katharina HERWIG, Hamburg (DE); Andreas BLECKMANN, Ahrensburg (DE); Petra KOCH, Hamburg (DE); Katharina ROPETER, Hamburg (DE); Theresa BEHRENDT, Hamburg (DE); Keti SCHUERINGS, Hamburg (DE)

(21) Appl. No.: 18/273,581

(22) PCT Filed: Jan. 13, 2022
(86) PCT No.: PCT/EP2022/050583

§ 371 Date: Jul. 21, 2023

(30) Foreign Application Priority Data
Jan. 25, 2021 (DE) 10 2021 200 621.3

(51) Int. Cl. A61K 8/39; A61K 8/06; A61K 8/23; A61K 8/365; A61K 8/37; A61K 8/92; A61Q 19/00

(52) U.S. Cl. CPC A61K 8/39; A61K 8/064; A61K 8/23; A61K 8/365; A61K 8/37; A61K 8/922; A61Q 19/00; A61K 2800/10; A61K 2800/30

(57) ABSTRACT

Cosmetic water-in-oil emulsion (W/O emulsion) containing
a) polyglyceryl-4 diisostearate/polyhydroxystearate/sebacate and
b) hydrogenated castor oil (INCI: Hydrogenated Castor Oil), wherein the preparation is free from mineral oil, paraffin wax, microcrystalline wax, shellac wax and polyethylene wax, free from polyacrylates, acrylate/C10-C30 alkyl acrylate crosspolymers and vinylpyrrolidone/hexadecene copolymers, and free from 3-(4-methylbenzylidene)camphor, 2-hydroxy-4-methoxybenzophenone (INCI: Oxybenzone), 2-ethylhexyl 4-methoxycinnamate (INCI Octyl Methoxycinnamate), ethylhexyl 2-cyano-3,3-diphenylacrylate (INCI: Octocrylene), parabens (particularly methyl, propyl and butyl paraben), methylisothiazolinone, chloromethylisothiazolinone and DMDM hydantoin, polyethylene glycol ethers or polyethylene glycol esters.

The present invention relates to a cosmetic water-in-oil emulsion (W/O emulsion) containing polyglyceryl-4 diisostearate/polyhydroxystearate/sebacate and hydrogenated castor oil (INCI: Hydrogenated Castor Oil), wherein the preparation is free from mineral oil, paraffin wax, microcrystalline wax, shellac wax and polyethylene wax, free from polyacrylates, acrylate/C10-C30 alkyl acrylate crosspolymers and vinylpyrrolidone/hexadecene copolymers, and free from 3-(4-methylbenzylidene)camphor, 2-hydroxy-4-methoxybenzophenone (INCI: Oxybenzone), 2-ethylhexyl 4-methoxycinnamate (INCI Octyl Methoxycinnamate), ethylhexyl 2-cyano-3,3-diphenylacrylate (INCI: Octocrylene), parabens (particularly methyl, propyl and butyl paraben), methylisothiazolinone, chloromethylisothiazolinone and DMDM hydantoin, polyethylene glycol ethers or polyethylene glycol esters.

The desire to look beautiful and attractive is naturally rooted in humans. Although ideals of beauty have changed over time, the pursuit of a flawless appearance has always been aimed for by humans. An essential part of a beautiful and attractive appearance is the condition and complexion of the skin.

In order for the skin to be able to perform the full range of its biological functions, it requires regular cleansing and care. Skin care products, generally creams, ointments or lotions, mostly serve for moisturizing and refatting the skin. Active ingredients are commonly added thereto, which are intended to regenerate the skin and for example to prevent and reduce the premature aging thereof (e.g., the appearance of fine lines and wrinkles).

In addition to cleansing and caring for the skin, cosmetics also have an aesthetic function. They are intended to “improve” the external appearance of the user according to the respective cultural ideas. Cosmetics thus fulfil a psychological and social function, since they increase the (visual) attractiveness of the user. This area includes above all “decorative” cosmetics, which change the appearance of the user with the help of colorants applied to the skin. Indirectly, however, cleansing and care products also have a positive influence, since clean, healthy skin corresponds to people's ideal of beauty.

Recently there has been an increasing trend towards “natural” cosmetics, the ingredients of which should if possible no longer come from petroleum products or be chemically synthesized. This trend overlaps nowadays with the trend towards “vegan” products. The search for alternative ingredients that meet these criteria poses particular challenges for product developers. This is because the replacement of known ingredients such as mineral oils, silicone oils and polyacrylates is practically always at the expense of disadvantages with respect to the product properties. The preparations become unstable and sensorially unattractive, which is unpleasantly noticeable, for example, when spreading the preparation on the skin and the lack of absorption capacity. These problems occur in particular with water-in-oil emulsions (W/O emulsions), which have recently enjoyed a resurgence in popularity. For instance, W/O emulsions without mineral oils and mineral waxes and without polyethylene glycol ethers or esters (so-called PEG derivatives) tend to be more unstable. During longer storage periods, particularly at higher temperatures, phase separations in the form of oil and/or water separation rapidly occur, which cannot easily be compensated for by a higher emulsifier content.

It was therefore the object of the present invention to develop a W/O emulsion without mineral oils and waxes and without polyethylene glycol ethers and esters, which is storage-stable even at higher temperatures and exhibits no (or significantly reduced) oil and water separation. In addition, the W/O emulsion should be applicable to various oil phase compositions, i.e., to ensure a wide range of applications in terms of the formula composition and be sensorially appealing (non-greasy, non-sticky, easily absorbed into the skin).

Surprisingly, the objects are achieved by a cosmetic water-in-oil emulsion (W/O emulsion) containing
a) polyglyceryl-4 diisostearate/polyhydroxystearate/sebacate and
b) hydrogenated castor oil (INCI: Hydrogenated Castor Oil), wherein the preparation is free from mineral oil, paraffin wax, microcrystalline wax, shellac wax and polyethylene wax, free from polyacrylates, acrylate/C10-C30 alkyl acrylate crosspolymers and vinylpyrrolidone/hexadecene copolymers, and free from 3-(4-methylbenzylidene)camphor, 2-hydroxy-4-methoxybenzophenone (INCI: Oxybenzone), 2-ethylhexyl 4-methoxycinnamate (INCI Octyl Methoxycinnamate), ethylhexyl 2-cyano-3,3-diphenylacrylate (INCI: Octocrylene), parabens (particularly methyl, propyl and butyl paraben), methylisothiazolinone, chloromethylisothiazolinone and DMDM hydantoin, polyethylene glycol ethers or polyethylene glycol esters.

Although DE 102017221672 and DE 102018217130 are known to those skilled in the art, these documents could not have indicated the approach of the present invention.

It is preferred according to the invention if the preparation contains hydrogenated castor oil (INCI: Hydrogenated Castor Oil) at a concentration of 0.3 to 1.0% by weight, based on the total weight of the preparation.

Embodiments of the present invention that are advantageous according to the invention are characterized in that the preparation contains diisostearoyl polyglyceryl-3 dimer dilinoleate. It is also advantageous according to the invention if the preparation contains polyglyceryl-4 diisostearate/polyhydroxystearate/sebacate at a concentration of 0.2 to 2.0% by weight, based on the total weight of the preparation, with a concentration of 0.5 to 1.5% by weight, based on the total weight of the preparation, being preferred according to the invention.

If the W/O emulsion according to the invention contains diisostearoyl polyglyceryl-3 dimer dilinoleate, it is advantageous according to the invention if the preparation contains diisostearoyl polyglyceryl-3 dimer dilinoleate at a concentration of 0.2 to 2.0% by weight, based on the total weight of the preparation. Particular preference is given to a concentration of 0.5 to 1.5% by weight.

In addition, it is advantageous in the context of the present invention if the preparation contains one or more oils selected from the group of triglyceride compounds. The advantageous use concentration according to the invention for these triglycerides (total amount of triglycerides) is from 0.5 to 5.0% by weight, particularly preferably 0.75 to 4.5% by weight, based on the total weight of the preparation.

It is preferred according to the invention if the preparation contains coco-caprylate/caprate (INCI: Coco-Caprylate/Caprate). The advantageous use concentration according to the invention for coco-caprylate/caprate (INCI: Coco-Caprylate/Caprate) is from 0.5 to 5.0% by weight, based on the total weight of the preparation. Particular preference is given to the concentration range from 0.75 to 4.5% by weight, based on the total weight of the preparation.

Furthermore, embodiments of the present invention that are advantageous according to the invention are characterized in that the preparation contains one or more palmitates.

In particular, it is advantageous according to the invention if the preparation contains cetyl palmitate and/or isopropyl palmitate.

If the preparation contains cetyl palmitate, this component is advantageously used according to the invention at a concentration of 0.2 to 1% by weight, based on the total weight of the preparation. Particular preference is given to a concentration of 0.3 to 0.8% by weight.

If the preparation contains isopropyl palmitate, this component is advantageously used according to the invention at a concentration of 1.0 to 15.0% by weight, based on the total weight of the preparation. Particular preference is given to 3 to 12% by weight.

In addition, it is advantageous according to the invention if the preparation contains shea butter and/or sunflower wax (INCI: Helianthus Annuus Seed Cera).

If the preparation contains shea butter, this component is advantageously used according to the invention at a concentration of 0.5 to 3.0% by weight, particularly preferably at a concentration of 0.75 to 2.5% by weight, based on the total weight of the preparation.

If the preparation contains sunflower wax (INCI: Helianthus Annuus Seed Cera), this component is advantageously used according to the invention at a concentration of 0.1 to 0.75% by weight, particularly preferably at a concentration of 0.1 to 0.5% by weight, based on the total weight of the preparation.

Advantageous embodiments of the present invention are further characterized in that the preparation contains one or more plant oils.

It is preferred according to the invention if the preparation contains one or more oils selected from the group of almond oil compounds (INCI: Prunus Amygdalus Dulcis Oil) and/or sunflower oil (INCI: Helianthus Annuus Seed Oil).

If the preparation contains almond oil (INCI: Prunus Amygdalus Dulcis Oil), this component is advantageously used according to the invention at a concentration of 0.1 to 0.5% by weight, based on the total weight of the preparation.

If the preparation contains sunflower oil (INCI: Helianthus Annuus Seed Oil), this component is advantageously used according to the invention at a concentration of 0.1 to 0.5% by weight, based on the total weight of the preparation.

Embodiments of the present invention that are advantageous according to the invention are further characterized in that the preparation contains magnesium sulfate. According to the invention, magnesium sulfate is preferably added at a concentration of 0.3 to 3.0% by weight, preferably at a concentration of 0.5 to 2.0% by weight, based on the total weight of the preparation.

Embodiments of the present invention that are advantageous according to the invention are also characterized in that the preparation contains potassium sorbate.

If the preparation contains potassium sorbate, this component is advantageously used according to the invention at a concentration of 0.05 to 0.5% by weight, preferably at a concentration of 0.075 to 0.30% by weight, based on the total weight of the preparation.

Embodiments of the present invention that are advantageous according to the invention are also characterized in that the preparation contains a mixture of citric acid and sodium citrate.

If the preparation contains citric acid, this component is advantageously used according to the invention at a concentration of 0.05 to 0.25% by weight, based on the total weight of the preparation. This is then partially neutralized by adding NaOH, resulting in a citric acid/citrate buffer system.

According to the invention, a combination of potassium sorbate and citric acid/sodium citrate is particularly preferred.

In addition, the W/O emulsion according to the invention may contain further ingredients.

For example, further plant oils from the group of the compounds Persea gratis sima oil, Orbignya oleifera seed oil, Argania spinosa kernel oil, Prunus armeniaca kernel oil, Simmondsia chinensis seed oil, Cocos nucifera oil, Silybum marianum seed oil, Oenothera biennis oil, Olea europaea fruit oil, Vitis vinifera seed oil, Cannabis sativa seed oil, Olus oil, vegetable oil, Gossypium herbaceum seed oil, Arctium lappa seed oil, Macadamia ternifolia seed oil, Macadamia integrifolia seed oil, Zea mays germ oil, Prunus amygdalus dulcis oil, Ricinus communis seed oil, glycine soja oil, Helianthus annuus hybrid oil, Sesamum indicum seed oil, Brassica campestris seed oil.

The preparation may also contain one or more esters of caprylic acid and/or capric acid.

In such a case, it is preferred according to the invention if the esters of caprylic acid and/or capric acid are selected from the group of the compounds caprylic acid/capric acid triglycerides (INCI: Caprylic/Capric Triglyceride), Caprylyl caprylate/caprate (INCI: Caprylyl Caprylate/Caprate), Cocoglycerides (INCI: Coco Glycerides), ethylhexyl cocoate (INCI: Ethylhexyl Cocoate), decyl cocoate (INCI: Decyl Cocoate), isoamyl cocoate (INCI: Isoamyl Cocoate), dicaprylyl carbonate (INCI: Dicaprylyl Carbonate), coco-caprylate (INCI: Coco-caprylate).

It is advantageous in the context of the present invention if the preparation contains waxes and/or fatty alcohols. According to the invention, preference is given to using waxes or fatty alcohols from the list of the following compounds:
cetearyl alcohol, cetyl alcohol, stearyl alcohol, methyl palmitate, myristyl myristate, hydrogenated coco-glycerides, myristyl alcohol, behenyl alcohol, Theobroma cacao seed butter, hydrogenated rapeseed oil, caprylic/capric/myristic/stearic triglyceride, hydrogenated vegetable oil, carnauba wax, candelilla wax, tristearin.

The preparation may also contain one or more lipophilic components selected from the group of the compounds dicaprylyl ether, decyl oleate, octyldodecanol, squalane, triisostearin, shea butter ethyl ester, triheptanoin, hexyldecyl stearate, isoamyl laurate.

Embodiments of the present invention that are advantageous according to the invention are characterized in that the preparation contains one or more compounds selected from the group of the compounds alpha-lipoic acid, folic acid, phytoene, D-biotin, coenzyme Q10, alpha-glucosylrutin, carnitine, carnosine, natural isoflavonoids, flavonoids, creatine, creatinine, taurine, β-alanine, tocopherol, panthenol, magnolol, honokiol, glycerylglucose, hyaluronic acid and/or salts thereof, and/or licochalcone A.

In particular, it is advantageous according to the invention if the W/O emulsion contains glycerin.

Comparative Experiment

The following formulations were prepared, and their stability compared.

| Example formulations | Formulation 1 | Formulation 2 |
|---|---|---|
| INCI | Weight [%] | Weight [%] |
| Perfume | 0.5 | 0.5 |
| Prunus Amygdalus Dulcis Oil | 0.4 | 0.4 |
| Glycerin | 5.0 | 5.0 |
| Citric Acid | 0.09 | 0.09 |
| Sodium Citrate + Aqua | 0.17 | 0.17 |
| Potassium sorbate | 0.20 | 0.20 |
| Tocopherol | 0.06 | 0.06 |
| Aqua | to 100 | to 100 |
| Magnesium sulfate | 1.5 | 1.5 |
| Isopropyl palmitate | 10 | 10 |
| Vegetable oil | 1.0 | 1.0 |
| Cetyl palmitate | 0.3 | 0.3 |
| Butyrospermum Parkii Butter | 1.7 | 1.7 |
| Helianthus Annuus Seed Cera + Ascorbyl Palmitate + | 0.3 | 0.3 |
| Tocopherol + Helianthus Annuus Seed Oil | | |
| Isopropyl Stearate | 2.5 | 2.5 |
| Hydrogenated Castor Oil | 0.5 | 0 |
| Coco-Caprylate/Caprate | 1.0 | 1.0 |
| Ethylhexyl Cocoate | 2.5 | 2.5 |
| Polyglyceryl-4 Diisostearate/Polyhydroxystearate/Sebacate | 1.3 | 1.3 |
| Diisostearoyl Polyglyceryl-3 Dimer Dilinoleate | 1.0 | 1.0 |

No difference is noticeable immediately after preparation.

The formulations are stored at 40° C. Even after 30 days of storage, a slight oil separation can be seen in formulation 2. This instability becomes more and more evident after further storage. In the case of formulation 1, no instability can be seen over the course of storage.

### EXAMPLES

The examples below are intended to illustrate the present invention without limiting it. Unless otherwise stated, all quantitative data, fractions, and percentages are based on the weight and the total amount or on the total weight of the preparations.

| Example formulations | 1 | 2 | 3 | 4 | 5 |
|---|---|---|---|---|---|
| INCI | Weight [%] | Weight [%] | Weight [%] | Weight [%] | Weight [%] |
| Perfume | 0.2 | 0.7 | 0.3 | 0.4 | 0.5 |
| Prunus Amygdalus Dulcis Oil | 0.2 | 0.5 | 0.1 | 0.3 | 0.4 |
| Glycerin | 3.0 | 5.0 | 10.0 | 6.0 | 5.0 |
| Citric Acid | 0.09 | 0.09 | 0.09 | 0.09 | 0.09 |
| Sodium Citrate + Aqua | 0.17 | 0.17 | 0.17 | 0.17 | 0.17 |
| Potassium sorbate | 0.15 | 0.075 | 0.30 | 0.10 | 0.20 |
| Tocopherol | 0.05 | 0.10 | 0.07 | 0.15 | 0.06 |
| Aqua | to 100 | to 100 | to 100 | to 100 | to 100 |
| Magnesium sulfate | 0.5 | 1.5 | 1.0 | 0.8 | 1.5 |
| Isopropyl Palmitate | 0 | 10.0 | 6.0 | 5.0 | 11.0 |
| Plant Oil | 3.0 | 2.0 | 2.0 | 3.5 | 1.0 |
| Cetyl Palmitate | 0.6 | 0.5 | 0.4 | 0.8 | 0.3 |
| Butyrospermum Parkii Butter | 1.5 | 1.0 | 2.2 | 0.9 | 1.7 |
| Helianthus Annuus Seed Cera + Ascorbyl | 0.3 | 0.3 | 0.3 | 0.3 | 0.3 |
| Palmitate + Tocopherol + Helianthus | | | | | |
| Annuus Seed Oil | | | | | |
| Isopropyl Stearate | 4.0 | 3.0 | 3.5 | 4.5 | 2.5 |
| Hydrogenated Castor Oil | 0.5 | 0.3 | 1.0 | 0.4 | 0.6 |
| Coco-Caprylate/Caprate | 5.0 | 3.0 | 3.5 | 1.0 | 0 |
| Cocos Nucifera Oil | 3 | 0 | 0.5 | 2.5 | 0 |
| Ethylhexyl Cocoate | 3 | 0 | 1 | 0 | 2.5 |
| Polyglyceryl-4 | 0.8 | 1.2 | 1.5 | 1.0 | 1.3 |
| Diisostearate/Polyhydroxystearate/ | | | | | |
| Sebacate | | | | | |
| Diisostearoyl Polyglyceryl-3 Dimer | | | | | |
| Dilinoleate | 1.4 | 0.7 | 0 | 0.8 | 1.0 |
| Caprylic/Capric Triglyceride | 0 | 0 | 0 | 1.0 | 0 |

1.-15. (canceled)

16. A cosmetic water-in-oil (W/O) emulsion, wherein the emulsion comprises
(a) polyglyceryl-4 diisostearate/polyhydroxystearate/sebacate and
(b) hydrogenated castor oil,
the emulsion being free from mineral oil, paraffin wax, microcrystalline wax, shellac wax, polyethylene wax, polyacrylates, acrylate/C10-C30 alkyl acrylate crosspolymers, vinylpyrrolidone/hexadecene copolymers, 3-(4-methylbenzylidene)camphor, 2-hydroxy-4-methoxybenzophenone, 2-ethylhexyl 4-methoxycinnamate, ethylhexyl 2-cyano-3,3-diphenylacrylate, parabens, methylisothiazolinone, chloromethylisothiazolinone, DMDM hydantoin, polyethylene glycol ethers, polyethylene glycol esters.

17. The cosmetic W/O emulsion of claim 16, wherein the emulsion comprises from 0.3% to 1.0% by weight of (b), based on a total weight of the emulsion.

18. The cosmetic W/O emulsion of claim 16, wherein the emulsion further comprises diisostearoyl polyglyceryl-3 dimer dilinoleate.

19. The cosmetic W/O emulsion of claim 16, wherein the emulsion comprises from 0.2% to 2.0% by weight of (a), based on a total weight of the emulsion.

20. The cosmetic W/O emulsion of claim 19, wherein the emulsion comprises from 0.5% to 1.5% by weight of (a).

21. The cosmetic W/O emulsion of claim 17, wherein the emulsion comprises from 0.2% to 2.0% by weight of (a), based on a total weight of the emulsion.

22. The cosmetic W/O emulsion of claim 18, wherein the emulsion comprises from 0.2% to 2.0% by weight of diisostearoyl polyglyceryl-3 dimer dilinoleate, based on a total weight of the emulsion.

23. The cosmetic W/O emulsion of claim 22, wherein the emulsion comprises from 0.5% to 1.5% by weight of diisostearoyl polyglyceryl-3 dimer dilinoleate.

24. The cosmetic W/O emulsion of claim 16, wherein the emulsion further comprises one or more triglycerides.

25. The cosmetic W/O emulsion of claim 16, wherein the emulsion further comprises coco-caprylate/caprate.

26. The cosmetic W/O emulsion of claim 16, wherein the emulsion further comprises one or more palmitates.

27. The cosmetic W/O emulsion of claim 26, wherein the emulsion comprises one or both of cetyl palmitate and isopropyl palmitate.

28. The cosmetic W/O emulsion of claim 16, wherein the emulsion further comprises shea butter and/or sunflower wax.

29. The cosmetic W/O emulsion of claim 16, wherein the emulsion further comprises one or more plant oils.

30. The cosmetic W/O emulsion of claim 29, wherein the emulsion further comprises almond oil and/or sunflower oil.

31. The cosmetic W/O emulsion of claim 16, wherein the emulsion further comprises magnesium sulfate.

32. The cosmetic W/O emulsion of claim 16, wherein the emulsion further comprises potassium sorbate.

33. The cosmetic W/O emulsion of claim 16, wherein the emulsion further comprises a mixture of citric acid and sodium citrate.

34. A cosmetic water-in-oil (W/O) emulsion, wherein the emulsion comprises, based on a total weight of the emulsion,
from 0.2% to 2.0% by weight of
(a) polyglyceryl-diisostearate/polyhydroxystearate/sebacate,
from 0.3% to 1.0% by weight of
(b) hydrogenated castor oil, and
from 0.2% to 2.0% by weight of
(c) diisostearoyl polyglyceryl-3 dimer dilinoleate,
the emulsion being free from mineral oil, paraffin wax, microcrystalline wax, shellac wax, polyethylene wax, polyacrylates, acrylate/C10-C30 alkyl acrylate crosspolymers, vinylpyrrolidone/hexadecene copolymers, 3-(4-methylbenzylidene)camphor, 2-hydroxy-4-methoxybenzophenone, 2-ethylhexyl 4-methoxycinnamate, ethylhexyl 2-cyano-3,3-diphenylacrylate, parabens, methylisothiazolinone, chloromethylisothiazolinone, DMDM hydantoin, polyethylene glycol ethers, polyethylene glycol esters.

35. The cosmetic W/O emulsion of claim 34, wherein the emulsion comprises from 0.5% to 1.5% by weight of (a) and from 0.5% to 1.5% by weight of (c).
